(19) United States
(12) Patent Application Publication
(10) Pub. No.: US 2017/0326194 A1
(43) Pub. Date: Nov. 16, 2017

(54) THERAPEUTIC COMPOSITIONS

(71) Applicant: CAPE KINGDOM NUTRACEUTICALS (PTY) LTD, Big Bay (ZA)

(72) Inventor: Michael Duncombe STANDER, Big Bay (ZA)

(21) Appl. No.: 15/532,417

(22) PCT Filed: Dec. 1, 2015
(86) PCT No.: PCT/IB2015/059237

§ 371 Date: Jun. 1, 2017

(30) Foreign Application Priority Data
Dec. 1, 2014 (ZA) 2014/08791

(51) Int. Cl. A61K 36/75

(52) U.S. Cl. CPC A61K 36/75

(57) ABSTRACT

The invention discloses a therapeutic composition, which includes at least one active ingredient originating from an aqueous Buchu extract and/or Buchu oil (betulina agathosma) or bioactive fraction thereof in a pharmaceutically acceptable form. The composition includes an effective amount of one or more anti-bacterial, anti-fungal, anti-inflammatory, anti-cancer, or other therapeutic active ingredients.

### FIELD OF INVENTION

The present invention relates to therapeutic compositions.

More particularly, the present invention relates to therapeutic compositions of Buchu plant material extracts.

### BACKGROUND TO INVENTION

Buchu is one of the best known medicinal plants of South Africa and is indigenous to the Cedarberg Mountains and surrounding areas. Despite its popularity little scientific evidence exists about the various medicinal uses of this small fynbos shrub from the family Rustaceae. The two primary species of Buchu used commercially are Agasthoma betulina (round-leaf Buchu) and Agathosma crenulata (oval-leaf Buchu). Besides its medicinal properties, Buchu oil is also used in the flavourant and fragrance industry, currently the largest commercial use thereof.

Buchu oil is typically prepared in a steam distillation process in which the Buchu oil required for the commercial market is extracted from the plant material and separated from the by-products of this steam distillation process.

It is an object of the invention to suggest a novel therapeutic compositions of Buchu plant material extracts.

### SUMMARY OF INVENTION

According to the invention, there is provided a therapeutic composition comprising at least one active ingredient originating from an aqueous Buchu extract and/or Buchu oil (betulina agathosma) or bioactive fraction thereof in a pharmaceutically acceptable form.

Preferably, the therapeutic composition is a pharmaceutical composition comprising a therapeutically effective amount of the at least one active ingredient and one or more pharmaceutically acceptable carriers or additives.

The invention extends to a modified aqueous Buchu extract and/or Buchu oil (betulina agathosma) or bioactive fraction thereof comprising an effective amount of one or more anti-bacterial, anti-fungal, anti-inflammatory, anti-cancer, or other therapeutic active ingredients.

The invention also extends to a therapeutic composition, pharmaceutical composition or modified aqueous Buchu extract and/or Buchu oil (betulina agathosma) or bioactive fraction thereof for use in a method of inducing an anti-bacterial, anti-fungal, anti-inflammatory, anti-cancer, or other therapeutic response in a mammal, preferably a human, in need thereof.

The invention extends further to the use of an aqueous Buchu extract and/or Buchu oil (betulina agathosma) or bioactive fraction thereof in the manufacture of a medicament for use in a method of inducing an anti-bacterial, anti-fungal, anti-inflammatory, anti-cancer, or other therapeutic response in a mammal, preferably a human, in need thereof.

According to a further aspect of the invention, there is provided a method of treating an ailment, infection or disease in a patient comprising administering to a patient in need thereof a therapeutically effective amount of at least one active ingredient obtained from an aqueous Buchu extract and/or Buchu oil (betulina agathosma) or bioactive fraction thereof.

According to yet another aspect of the invention, there is provided generally a method of preparing an aqueous Buchu extract and/or Buchu oil (betulina agathosma), including the steps of providing a source of Buchu plant material, preferably from the species Agasthoma betulina (round-leaf Buchu) and/or Agathosma crenulata (oval-leaf Buchu), treating the plant material in order to extract the water soluble components thereof into an aqueous portion, and treating the aqueous portion to remove any non-water-soluble components and/or other impurities.

The composition may be used to treat and/or inhibit and/or killing of the following bacterial strains and Fungus:
(a) MRSA;
(b) Pseudomonas aeruginosa;
(c) Shigella;
(d) Staphylococcus aureus (both methicillin and vancomycin resistant strains);
(e) Tuberculosis;
(f) Acinetobacter spp (particularly baumannii);
(g) Vancomycin resistant enterococci;
(h) Klebsiella pneumonia;
(i) Streptococcus pneumonia; and
(j) Malassezia pachydermatis (Fungus).

Preferably, the aqueous Buchu extract is further treated to obtain one or more bioactive fractions containing at least one active ingredient suitable for use in therapy. In particular, the aqueous Buchu extract is contacted with a suitable solvent to obtain various fractions thereof and/or the active ingredients directly.

Alternatively, the aqueous extract may be obtained from the water-based by-product(s) or residue(s) from a conventional Buchu oil preparation process, the water-based by-product(s) or residue(s) being further treated to recover the aqueous Buchu extract.

The solvent is preferably selected based on its polarity to extract active molecules from the aqueous Buchu extract. Preferred solvents include ethyl acetate (EtAc), dichloromethane (DCM) and chloroform (chlfm), chloroform being most preferred.

According to the invention, there is provided a therapeutic composition comprising a watery extract of at least one active ingredient originating from an aqueous Buchu extract or bioactive fraction thereof in a pharmaceutically acceptable form.

Preferably, the therapeutic composition is a pharmaceutical composition comprising a water extract of a therapeutically effective amount of at least one active ingredient and one or more pharmaceutically acceptable carriers or additives.

Also according to the invention a therapeutic composition of Buchu plant material extracts for treating Type 1 and Type 2 diabetes.

The invention extends to a modified aqueous Buchu extract or bioactive fraction thereof comprising an effective amount of one or more anti-diabetic therapeutic active ingredients.

The invention also extends to a watery extract therapeutic composition, pharmaceutical composition or modified aqueous Buchu extract or bioactive fraction thereof for use in a method of inducing an anti-diabetic therapeutic response in a mammal, preferably a human, in need thereof.

The invention extends further to the use of an aqueous Buchu extract or bioactive fraction thereof in the manufacture of a medicament for use in a method of inducing an anti-diabetic therapeutic response in a mammal, preferably a human, in need thereof.

According to a further aspect of the invention, there is provided a method of treating diabetes in a patient comprising administering to a patient in need thereof a therapeutically effective amount of at least one active ingredient obtained from an aqueous Buchu extract or bioactive fraction thereof.

According to yet another aspect of the invention, there is provided generally a method of preparing an aqueous Buchu extract, including the steps of providing a source of Buchu plant material, preferably from the species Agasthoma betulina (round-leaf Buchu) and/or Agathosma crenulata (oval-leaf Buchu), treating the plant material in order to extract the water soluble components thereof into an aqueous portion, and treating the aqueous portion to remove any non-water-soluble components and/or other impurities.

Preferably, the aqueous Buchu extract is further treated to obtain one or more bioactive fractions containing at least one active ingredient suitable for use in therapy. In particular, the aqueous Buchu extract is contacted with a suitable solvent to obtain various fractions thereof and/or the active ingredients directly.

Alternatively, the aqueous extract may be obtained from the water-based by-product(s) or residue(s) from a conventional Buchu oil preparation process, the water-based by-product(s) or residue(s) being further treated to recover the aqueous Buchu extract.

The solvent is preferably selected based on its polarity to extract active molecules from the aqueous Buchu extract. Preferred solvents include ethyl acetate (EtAc), dichloromethane (DCM) and chloroform (chlfm), chloroform being most preferred.

According to the invention, there is provided a therapeutic composition comprising a watery extract of at least one active ingredient originating from an aqueous Buchu extract or bioactive fraction thereof in a pharmaceutically acceptable form.

Preferably, the therapeutic composition is a pharmaceutical composition comprising a water extract of a therapeutically effective amount of at least one active ingredient and one or more pharmaceutically acceptable carriers or additives.

The invention extends to a modified aqueous Buchu extract or bioactive fraction thereof comprising an effective amount of one or more anti-hypertensive therapeutic active ingredients.

The invention also extends to a watery extract therapeutic composition, pharmaceutical composition or modified aqueous Buchu extract or bioactive fraction thereof for use in a method of inducing an anti-hypertensive therapeutic response in a mammal, preferably a human, in need thereof.

The invention extends further to the use of an aqueous Buchu extract or bioactive fraction thereof in the manufacture of a medicament for use in a method of inducing an anti-hypertensiven therapeutic response in a mammal, preferably a human, in need thereof.

According to a further aspect of the invention, there is provided a method of treating hypertension in a patient comprising administering to a patient in need thereof a therapeutically effective amount of at least one active ingredient obtained from an aqueous Buchu extract or bioactive fraction thereof.

According to yet another aspect of the invention, there is provided generally a method of preparing an aqueous Buchu extract, including the steps of providing a source of Buchu plant material, preferably from the species Agasthoma betulina (round-leaf Buchu) and/or Agathosma crenulata (oval-leaf Buchu), treating the plant material in order to extract the water soluble components thereof into an aqueous portion, and treating the aqueous portion to remove any non-water-soluble components and/or other impurities.

Preferably, the aqueous Buchu extract is further treated to obtain one or more bioactive fractions containing at least one active ingredient suitable for use in therapy. In particular, the aqueous Buchu extract is contacted with a suitable solvent to obtain various fractions thereof and/or the active ingredients directly.

Alternatively, the aqueous extract may be obtained from the water-based by-product(s) or residue(s) from a conventional Buchu oil preparation process, the water-based by-product(s) or residue(s) being further treated to recover the aqueous Buchu extract.

The solvent is preferably selected based on its polarity to extract active molecules from the aqueous Buchu extract. Preferred solvents include ethyl acetate (EtAc), dichloromethane (DCM) and chloroform (chlfm), chloroform being most preferred.

According to the invention, there is provided a veterinary therapeutic composition comprising at least one active ingredient originating from an aqueous Buchu extract and/or Buchu oil (betulina agathosma) or bioactive fraction thereof in a pharmaceutically acceptable form.

Preferably, the veterinary therapeutic composition is a pharmaceutical composition comprising a therapeutically effective amount of the at least one active ingredient and one or more pharmaceutically acceptable carriers or additives.

The composition may be used to treat dog ears.

The composition may be applied to dog ears.

The composition may be used to treat at least one the following bacterial strains and fungus:

(a) Malassezie pachydermatis; and
(b) Pseudonomus aeruginosa.

The invention extends to a modified aqueous Buchu extract and/or buchu oil (betulina agathosma) or bioactive fraction thereof comprising an effective amount of one or more anti-bacterial, anti-fungal, anti-inflammatory, anti-cancer, or other therapeutic active ingredients.

The invention also extends to a veterinary therapeutic composition, pharmaceutical composition or modified aqueous Buchu extract and/or buchu oil (betulina agathosma) or bioactive fraction thereof for use in a method of inducing an anti-bacterial, anti-fungal, anti-inflammatory, anti-cancer, or other therapeutic response in a mammal, preferably an animal, in need thereof.

The invention extends further to the use of an aqueous Buchu extract and/or Buchu oil (betulina agathosma) or bioactive fraction thereof in the manufacture of a medicament for use in a method of inducing an anti-bacterial, anti-fungal, anti-inflammatory, anti-cancer, or other therapeutic response in a mammal, preferably an animal, in need thereof.

According to a further aspect of the invention, there is provided a method of treating an ailment, infection or disease in an animal comprising administering to an animal in need thereof a therapeutically effective amount of at least one active ingredient obtained from an aqueous Buchu extract and/or buchu oil (betulina agathosma) or bioactive fraction thereof.

According to yet another aspect of the invention, there is provided generally a method of preparing an aqueous Buchu extract, including the steps of providing a source of Buchu plant material, preferably from the species Agasthoma betulina (round-leaf Buchu) and/or Agathosma crenulata (oval-leaf Buchu), treating the plant material in order to extract the water soluble components thereof into an aqueous portion, and treating the aqueous portion to remove any non-water-soluble components and/or other impurities.

Preferably, the aqueous Buchu extract is further treated to obtain one or more bioactive fractions containing at least one active ingredient suitable for use in therapy. In particular, the aqueous Buchu extract is contacted with a suitable solvent to obtain various fractions thereof and/or the active ingredients directly.

Alternatively, the aqueous extract may be obtained from the water-based by-product(s) or residue(s) from a conventional Buchu oil preparation process, the water-based by-product(s) or residue(s) being further treated to recover the aqueous Buchu extract.

The solvent is preferably selected based on its polarity to extract active molecules from the aqueous Buchu extract. Preferred solvents include ethyl acetate (EtAc), dichloromethane (DCM) and chloroform (chlfm), chloroform being most preferred.

### BRIEF DESCRIPTION OF DRAWINGS

The invention will now be described by way of example with reference to the accompanying schematic drawings.

In the drawings there is shown in:

FIG. 1 a chromatogram showing time fractions collected from an aqueous Buchu extract of the invention;

FIGS. 2 to 9 graphs of the anti-bacterial activity of the time fractions collected from an aqueous Buch extract of the invention;

FIGS. 10/11 graphs of the anti-fungal activity of the time fractions collected from an aqueous Buchu extract of the invention;

FIG. 12 a graph of the anti-inflammatory activity of the time fractions collected from an aqueous Buchu extract of the invention;

FIGS. 13/14 graphs indicating no significant effect on cytokine secretion of the time based fractions collected from an aqueous Buchu extract of the invention;

FIG. 15 a representative plot of the anti-cancer activity of aqueous Buchu water time-based extracts of the invention;

FIGS. 16/17 graphs of the anti-cancer activity of the time fractions collected from an aqueous Buchu extract of the invention;

FIG. 18: a graph showing blood glucose levels of Experiment 1 of a watery extract of Buchu according to the invention;

FIG. 19: a graph showing glucose tolerance levels of Experiment 1 of a watery extract of Buchu according to the invention;

FIG. 20: Glucose utilization by Chang liver cells;

FIG. 21: Glucose utilization bny 3t3-L1 cells;

FIG. 22: a graph showing the effects of both the HFD and the Buchu treatment of Experiment 3 of a watery extract of Buchu according to the invention; and

FIG. 23: a graph showing the body weight effect of a watery extract according to the invention.

### DETAILED DESCRIPTION OF DRAWINGS

According to the invention, there is provided a therapeutic composition comprising at least one active ingredient originating from an aqueous Buchu extract and/or Buchu oil (betulina agathosma) or bioactive fraction thereof in a pharmaceutically acceptable form.

Preferably, the therapeutic composition is a pharmaceutical composition comprising a therapeutically effective amount of the at least one active ingredient and one or more pharmaceutically acceptable carriers or additives.

The invention extends to a modified aqueous Buchu extract and/or Buchu oil (betulina agathosma) or bioactive fraction thereof comprising an effective amount of one or more anti-bacterial, anti-fungal, anti-inflammatory, anti-cancer, or other therapeutic active ingredients.

The invention also extends to a therapeutic composition, pharmaceutical composition or modified aqueous Buchu extract and/or Buchu oil (betulina agathosma) or bioactive fraction thereof for use in a method of inducing an anti-bacterial, anti-fungal, anti-inflammatory, anti-cancer, or other therapeutic response in a mammal, preferably a human, in need thereof.

The invention extends further to the use of an aqueous Buchu extract and/or Buchu oil (betulina agathosma) or bioactive fraction thereof in the manufacture of a medicament for use in a method of inducing an anti-bacterial, anti-fungal, anti-inflammatory, anti-cancer, or other therapeutic response in a mammal, preferably a human, in need thereof.

According to a further aspect of the invention, there is provided a method of treating an ailment, infection or disease in a patient comprising administering to a patient in need thereof a therapeutically effective amount of at least one active ingredient obtained from an aqueous Buchu extract and/or Buchu oil (betulina agathosma) or bioactive fraction thereof.

According to yet another aspect of the invention, there is provided generally a method of preparing an aqueous Buchu extract and/or Buchu oil (betulina agathosma), including the steps of providing a source of Buchu plant material, preferably from the species Agasthoma betulina (round-leaf Buchu) and/or Agathosma crenulata (oval-leaf Buchu), treating the plant material in order to extract the water soluble components thereof into an aqueous portion, and treating the aqueous portion to remove any non-water-soluble components and/or other impurities.

The composition can be used to treat and/or inhibit and/or killing of the following bacterial strains and Fungus:
(a) MRSA;
(b) Pseudomonas aeruginosa;
(c) Shigella;
(d) Staphylococcus aureus (both methicillin and vancomycin resistant strains);
(e) Tuberculosis;
(f) Acinetobacter spp (particularly baumannii);
(g) Vancomycin resistant enterococci;
(h) Klebsiella pneumonia;
(i) Streptococcus pneumonia; and
(j) Malassezia pachydermatis (Fungus).

Preferably, the aqueous Buchu extract is further treated to obtain one or more bioactive fractions containing at least one active ingredient suitable for use in therapy. In particular, the aqueous Buchu extract is contacted with a suitable solvent to obtain various fractions thereof and/or the active ingredients directly.

Alternatively, the aqueous extract may be obtained from the water-based by-product(s) or residue(s) from a conventional Buchu oil preparation process, the water-based by-product(s) or residue(s) being further treated to recover the aqueous Buchu extract.

The solvent is preferably selected based on its polarity to extract active molecules from the aqueous Buchu extract. Preferred solvents include ethyl acetate (EtAc), dichloromethane (DCM) and chloroform (chlfm), chloroform being most preferred.

Also according to the invention, there is provided a veterinary therapeutic composition comprising at least one active ingredient originating from an aqueous Buchu extract and/or Buchu oil (betulina agathosma) or bioactive fraction thereof in a pharmaceutically acceptable form.

Preferably, the veterinary therapeutic composition is a pharmaceutical composition comprising a therapeutically effective amount of the at least one active ingredient and one or more pharmaceutically acceptable carriers or additives.

The composition can be used to treat dog ears and can be applied to dog ears.

The composition can be used to treat at least one the following bacterial strains and fungus:

(a) Malassezie pachydermatis; and
(b) Pseudonomus aeruginosa.

As mentioned in the background to the invention, the oil extracted from Buchu plant material has traditionally been used in commercial therapeutic and other uses. It has now surprisingly been found that Buchu plant material includes a wealth of water soluble molecules or components that have been found to show various therapeutic properties including, but not necessarily exclusively, anti-bacterial, anti-fungal, anti-inflammatory and anti-cancer properties.

In order to test the bioactive nature of the water soluble components of Buchu plant material, the aqueous portion was obtained from a conventional steam distillation process traditionally used for extracting Buchu oil from Buchu plant material. The aqueous portion was separated from the oil portion, and treated in order to remove impurities and other non-water-soluble components, including Buchu oil residue. This modified aqueous Buchu extract, hereinafter “Buchu water” for convenience, was then treated in a further extraction process, as detailed more fully in what follows, in order to recover a number of bioactive fractions, including the bioactive molecules of interest.

Generally the Buchu water was contacted with various solvents, and the fractions were then analysed and their chemical profiles determined.

For testing purposes separate round-leaf and oval-leaf Buchu water samples were tested to identify chemical and biological differences. It is to be appreciated, however, that the preparation process extends to Buchu water obtained from a mixture of round-leaf Buchu and oval-leaf Buchu plant material.

In order to extract and concentrate the potential active molecules in Buchu water, and due to the aqueous nature of the product, it was determined that chemical extraction, or more particularly chemical fractionation, would be required.

Three different extraction solvents based on their polarity were used to extract the active molecules from round-leaf Buchu and oval-leaf Buchu water, respectively. This was done to determine which solvent yielded the best extraction profile based on its chemical profile. The extraction solvents used were ethyl acetate (EtAc), dichloromethane (DCM), and chloroform (chlfm).

Equal volumes of round-leaf Buchu water and oval-leaf Buchu water and extraction solvents were mixed in a separation funnel respectively and the solvent layer was collected. The solvent extraction step was repeated twice to ensure optimum extraction. The extract was then concentrated by removing the solvent by evaporation with a roto-evaporater. The residue was made up in a fixed volume of methanol after all the solvent was removed. The three different extracts were then appropriately stored for analytical analysis.

The different extracts were analysed by means of liquid chromatography with mass spectrometry detection (LC-MS). An optimized method and column type was established to show the differences between ethyl acetate, chloroform and dichloromethane solvents, and to show the differences in the extracts obtained from the round-leaf Buchu water and the oval-leaf Buchu water.

The results showed that the best suited extraction solvent is chloroform and that major differences occurred between the two species of Buchu. There were also minor regional and seasonal differences.

Having established that chloroform was the best solvent for extraction purposes the same extraction method was followed as described above. Sufficient volumes of the different round-leaf Buchu water and oval-leaf Buchu water were extracted with chloroform. The extracts were kept in their neat form instead of dissolving them in methanol. The extracts were kept separate according to leaf type and region and stored appropriately until further testing.

Instead of targeting each compound in the round-leaf Buchu extracts and oval-leaf Buchu extracts independently, a broad separation of the respective extracts based on the elution time from the liquid chromatography column was carried out. These time-based extracts each have specific chemical profiles with specific biological activities as compounds with similar chemical characteristics are grouped together in the same time-based extract. Compounds showing a specific biological activity can be isolated in this manner.

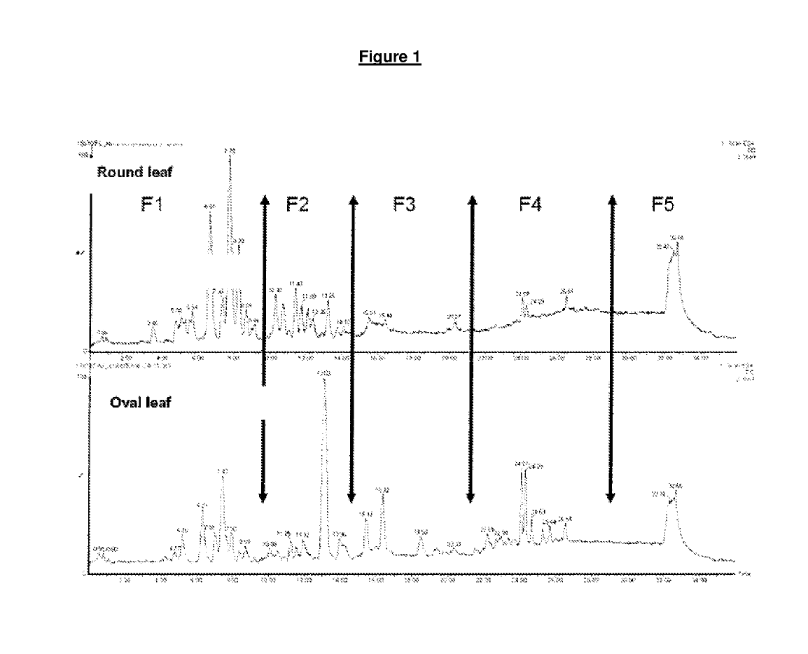

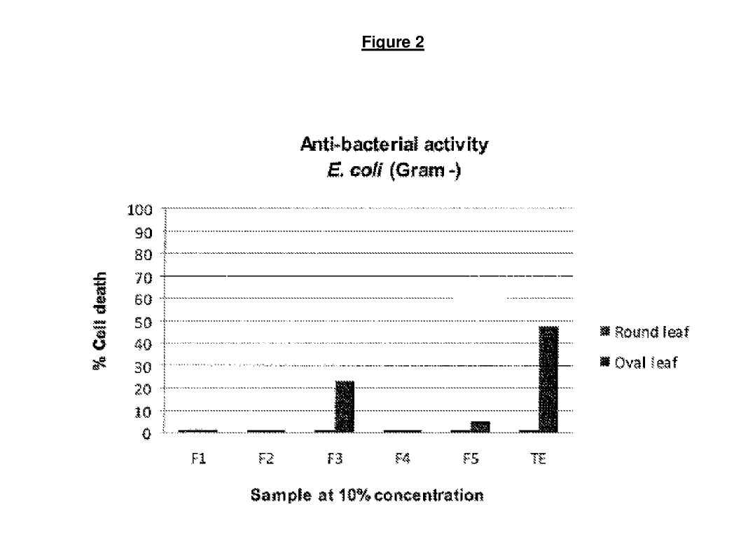

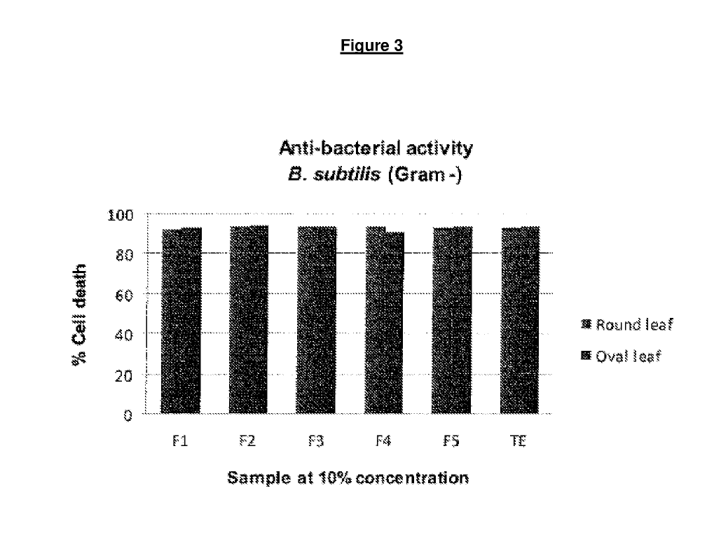

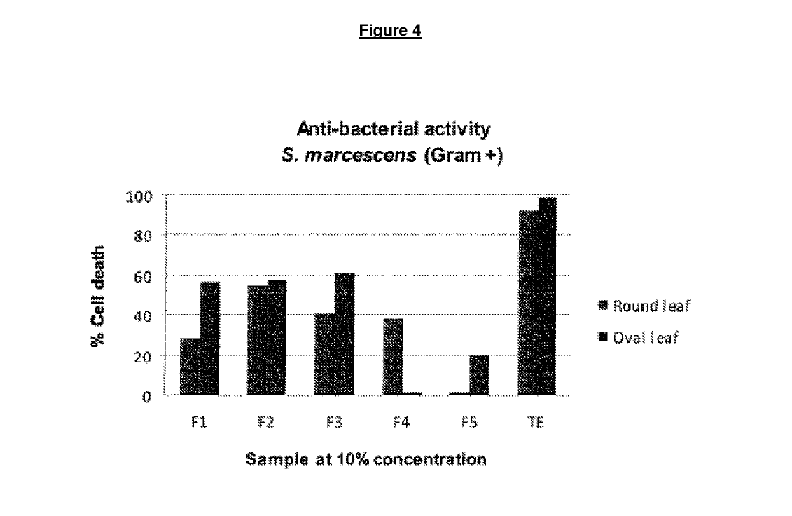

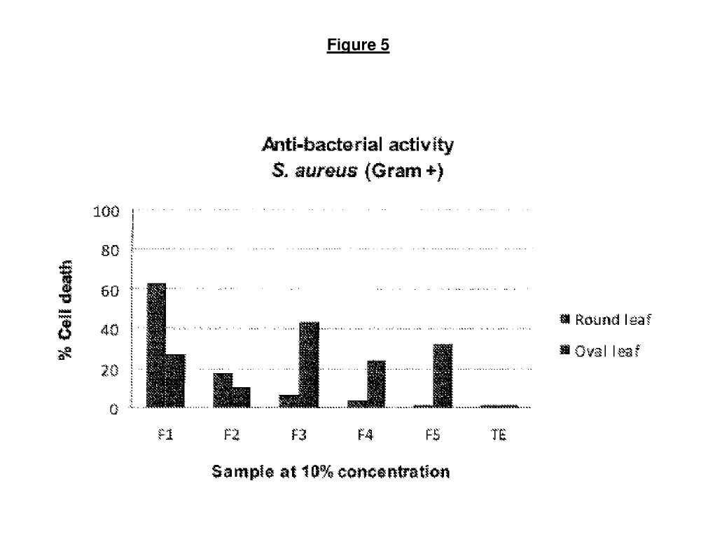

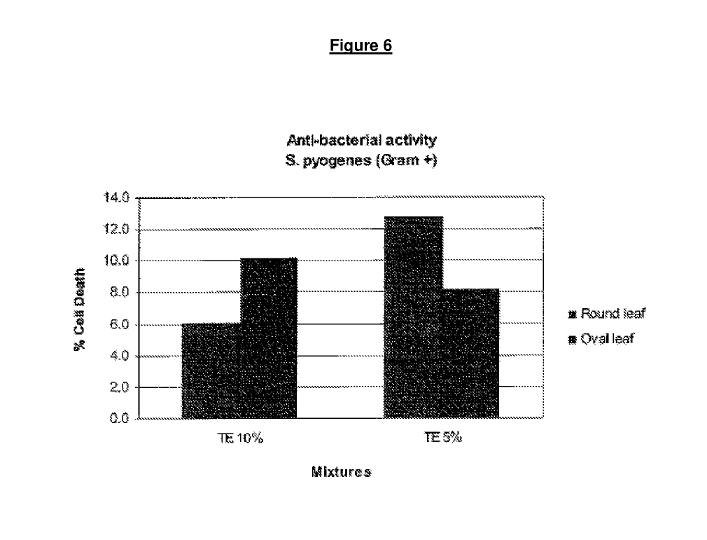

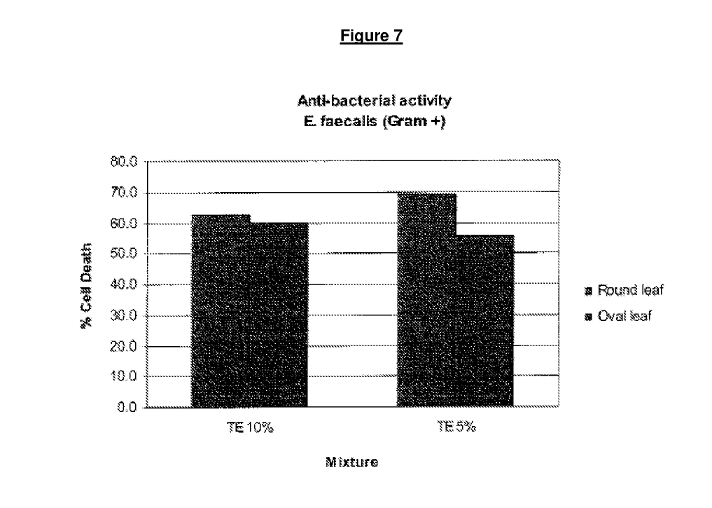

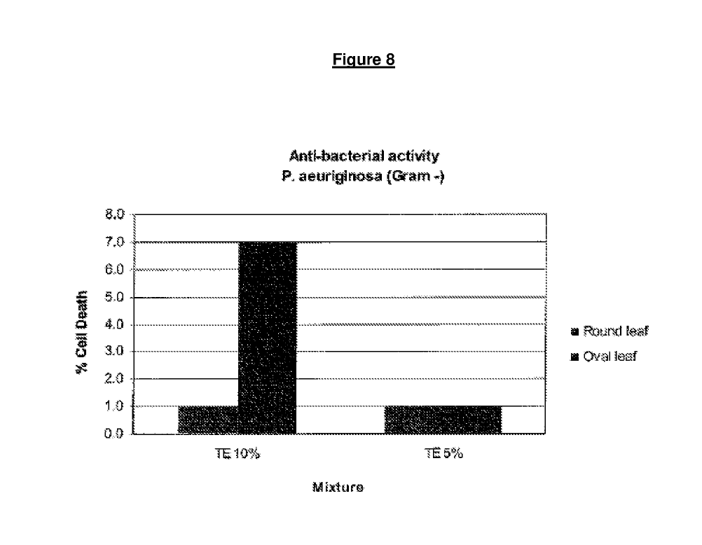

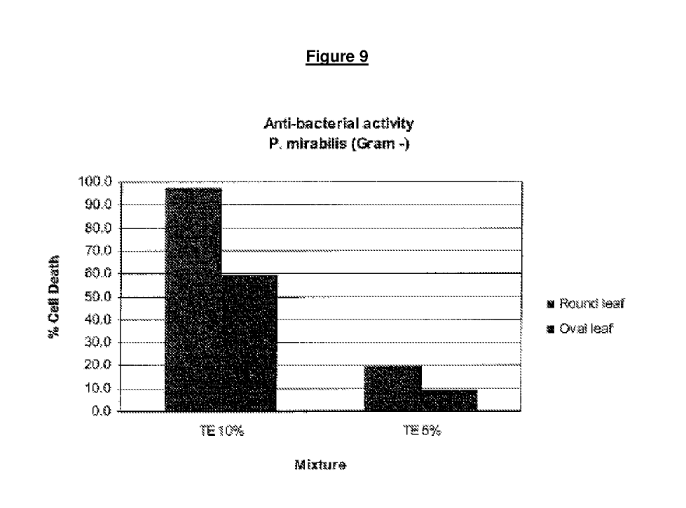

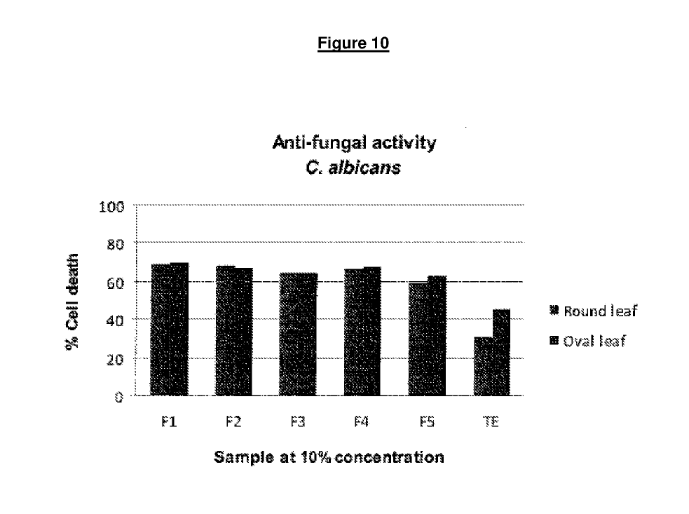

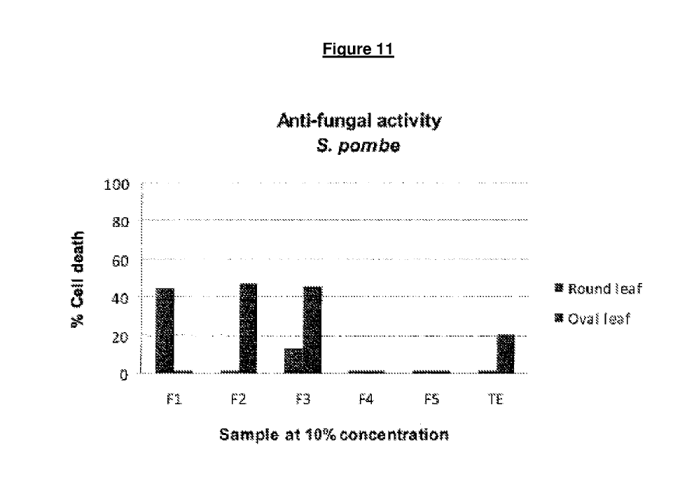

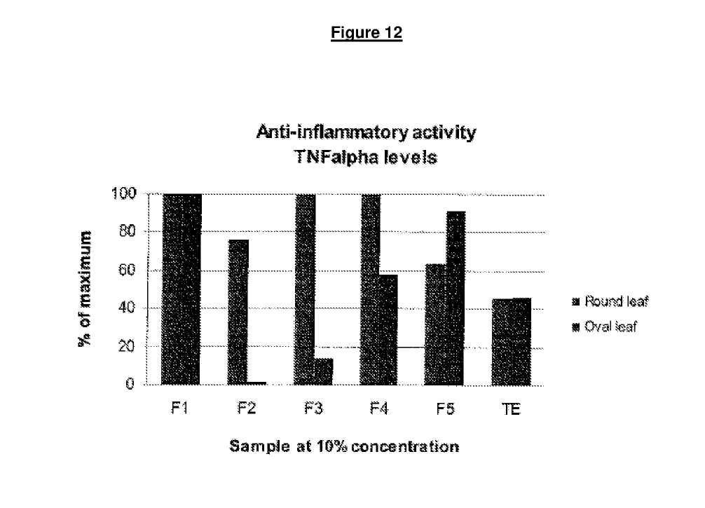

Extracts of the same leaf type prepared during the chloroform extraction of Buchu water were pooled together to yield an oval-leaf mix and a round-leaf mix, respectively. The collected time-based extractions are shown in accompanying FIG. 1.

Each time-based extract was DURPed in and back extracted with chloroform to produce a concentrate of that particular time-based extract. Furthermore, each time-based extract was made up in a specific volume of a 70% ethanol solution. This volume was calculated from the total volume yielded per time-based extract after time-based extraction and ensured that the relative concentration ratio of the compounds to each other remained the same when used in biological testing. These time-based extraction concentrates were stored appropriately for further testing.

A sample representing the total round-leaf extract and oval-leaf extract was also included in the biological testing. This sample was prepared from the round-leaf and oval-leaf mixed extracts used in the time extraction and was diluted so that the relevant concentration was more or less the same as that of the time-extractions since determining absolute concentrations was not possible at this stage.

Round-leaf time-extractions and oval-leaf time-extractions as well as total extract sample for each species were produced and coded as shown in Table 1:

TABLE 1

| Time-fraction | Buchu specie | Lab code |
|---|---|---|
| Fraction 1 | Round leaf | F1-R |
| Fraction 2 | Round leaf | F2-R |
| Fraction 3 | Round leaf | F3-R |
| Fraction 4 | Round leaf | F4-R |
| Fraction 5 | Round leaf | F5-R |
| Total extract | Round leaf | TE-R |
| Fraction 1 | Oval leaf | F1-O |
| Fraction 2 | Oval leaf | F2-O |
| Fraction 3 | Oval leaf | F3-O |
| Fraction 4 | Oval leaf | F4-O |
| Fraction 5 | Oval leaf | F5-O |
| Total extract | Oval leaf | TE-O |

Having obtained the abovementioned fractions, the biological activity of the Buchu water plant extracts was determined.

### 1. Anti-Bacterial Activity

The different extracts were tested at two different concentrations (10% and 5%) for activity against four different bacteria. These bacteria were Escherichia coli ATCC 25922 (Gram −), Bacillus subtilis ATCC21332 (Gram −), Staphylococcus aureus ATCC25723 (Gram+) and Serratia marcescens DMS 12481 (Gram+). Cultures of the bacteria were incubated overnight in the presence of the time-based extractions. A representative control was also included in each instance to establish a solvent background, thereby compensating for any possible interference from the 70% ethanol solution containing the diluted time-based extracts and total extracts. All results were compared to the representative controls.

An additional study was done to determine the effects of the total extracts at two different concentrations (10% and 5%) against 5 other bacteria. These were Staphylococcus pyogenes, Enterococcus faecalis, Pseudomonas aeuriginosa, Proteus mirabilis, and Methicillin resistant Staphylococcus aureus (MRSA). The study design was the same as mentioned above.

A chemiluminescent assay measuring ATP generation was used to determine the amount of viable bacteria cells in each instance. A higher number of bacterial cells will yield a higher concentration of ATP and vice versa.

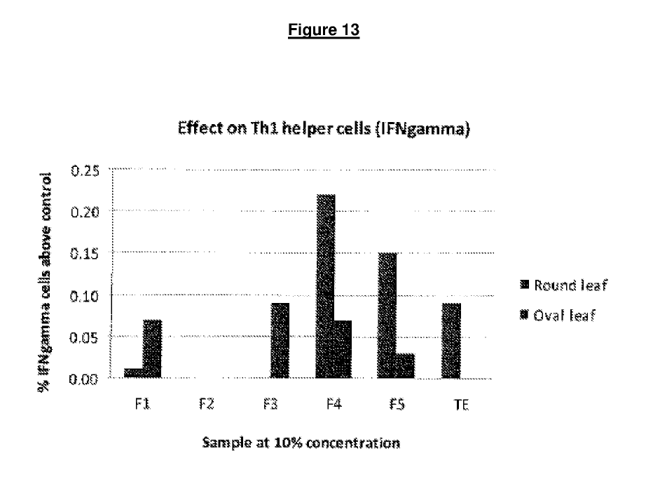

The results are shown in the graphs of accompanying FIGS. 2 to 9.

The graphs show activity on E. coli by Fraction 3 of the oval-leaf time-based extract, activity on B. subtilis by the total extracts of both the round-leaf time-based extract and the oval-leaf time-based extract, activity on S. marcescens by Fractions 1, 2 and 3 of the oval-leaf time-based extract and Fraction 2 of the round-leaf time-based extract, activity on S. aureus by Fraction 1 of the round-leaf time-based extract and Fraction 3 of the oval-leaf time-based extract. Both round-leaf and oval-leaf total time-based extracts have slight activity against S. pyogene and significant activity against E. faecalis and P. mirabilis (round-leaf time-based extract more potent). There was negligible activity against P. aeuriginosa and no activity against MRSA (data not shown).

### 2. Anti-Fungal Activity

The different time-based extracts were tested at two different concentrations (10% and 5%) for activity against two different yeasts. These two yeasts were Candida albicans and Schizosaccharomyces pombe IFO 0347. Cultures of the yeasts were incubated overnight in the presence of the time-based extracts. A representative control was also included in each instance to establish a solvent background, thereby compensating for any possible interference from the 70% ethanol solution in which the time-based extracts and total extracts were diluted. All results were compared to the representative controls.

The same chemiluminescent assay used for anti-bacterial detection was utilized to determine the amount of viable cells after incubation.

The findings are shown in the graphs of accompanying FIGS. 10 and 11.

The graphs show activity against C. albicans by most Fractions of round-leaf and oval-leaf time-based extracts and activity against S. pombe by Fraction 1 of the round-leaf time-based extract and Fractions 2 and 3 of the oval-leaf time based extracts.

### 3. Anti-Inflammatory Activity

An in vitro whole blood assay was set up where whole bloods from healthy volunteers were incubated for a set period in the presence of the different time-based extracts at two different concentrations (10% and 5% final concentration). At the same time of addition of the different time-based extracts an inflammatory response was induced in these samples via stimulation with an antigen. Corresponding controls were also set up for comparative purposes. In order to evaluate the possible anti-inflammatory effects of the different time-based extracts, the release of two of the major pro-inflammatory cytokines, IL-6 and TNF-α, were measured by means of enzyme-linked immunosorbent assay (ELISA). A decrease in one or both of these cytokines when compared to the relevant control will indicate an in virto anti-inflammatory activity.

The results are shown in the graph of accompanying FIG. 12. An anti-inflammatory effect will be portrayed by a decrease in the percentage of the maximum.

The graph shows an anti-inflammatory activity against TNF-α release by Fractions 2 and 3 of the oval-leaf time-based extracts.

### 4. Effects on Immune Cell Subsets

The effects of the different time-based extracts on important immune cell subsets were determined in vitro by flow cytometric methods. These subsets of T lymphocytes are known as T-helper cells (Th cells), consist of Th1 and Th2 cells and are responsible for cellular and humoral immunity, respectively. The in vitro assay used whole blood incubated overnight in the presence of the different time-based extracts at final concentrations of 10 and 5%. Stimulated whole blood as well as non-stimulated blood was also incubated overnight for comparative purposes. The overnight incubated samples were then analyzed on a flow cytometer for intracellular cytokines specific for the two different subsets of T lymphocytes. These cytokines are interferon-gamma (IFN-γ) produced by Th1 cells and interleukin-4 (IL-4) produced by Th2 cells.

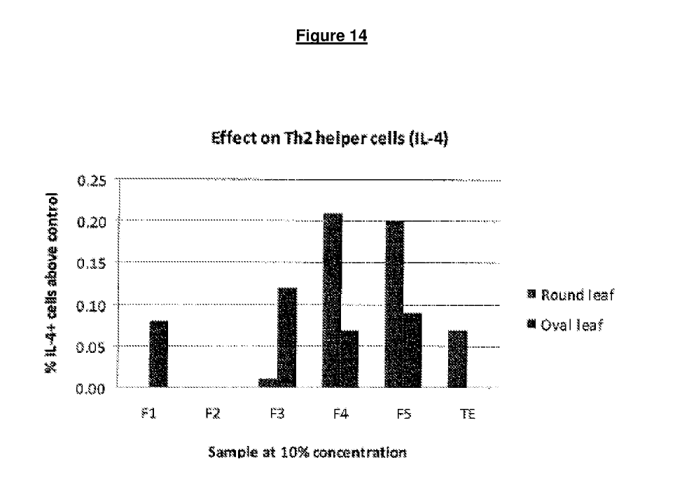

The results are shown in the graphs of accompanying FIGS. 13 and 14. An effect favouring a cellular immune response will be depicted by an increase in IFN-γ positive cells while an effect favouring a humoral immune response will be depicted in an increase in IL-4 positive cells.

The graphs show that the time-based extracts had no significant effect on cytokine secretion.

### 5. Anti-Cancer Activity

The four cell lines used for testing the potential anti-cancer properties of the time-based extracts were Jurkat (T cell leukemia), MOLT-4 (lymphoblastic leukemia), K-562 (chronic myelogenous leukemia), and HL60 (acute promyelocytic leukemia). The time-based extracts were tested at final concentrations of 10 and 5%. The bio-assay involved flow cytometry based analysis of the amount of dead cells present after overnight incubation in the presence of the different time-based extracts at the above mentioned concentrations. The potential of each concentration of ionic Ag to induce cell death (cytotoxic) was determined by comparing the amount of dead cells to that of a corresponding control sample (sample containing the same volume of 70% ethanol as the samples with the Buchu time-based extracts).

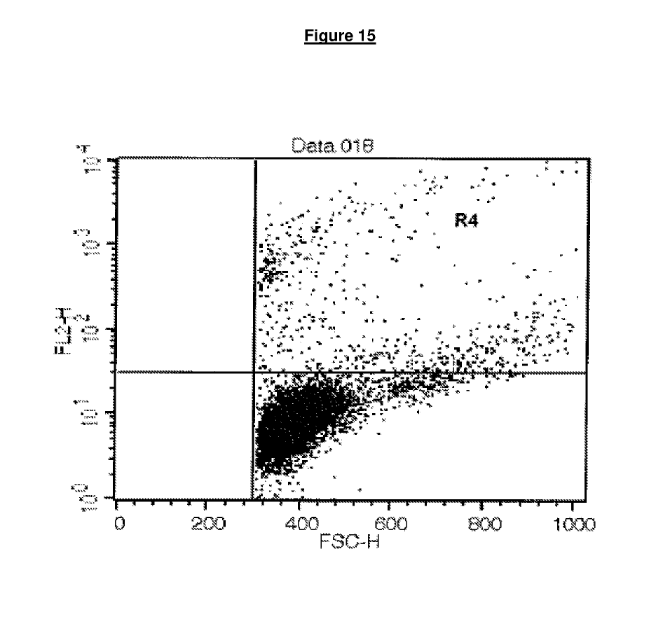

A representative plot of the results is shown in FIG. 15. If the fractions are able to induce direct cell death after overnight incubation, the cells would be measured in region R4 due to the uptake of the dye used (PI).

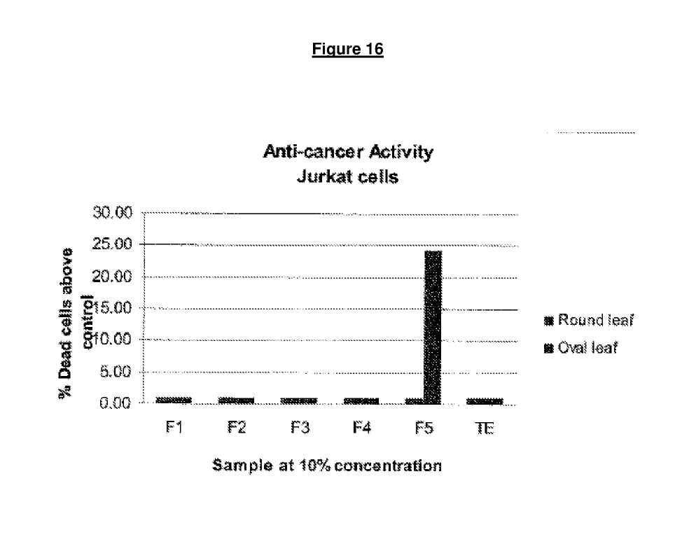

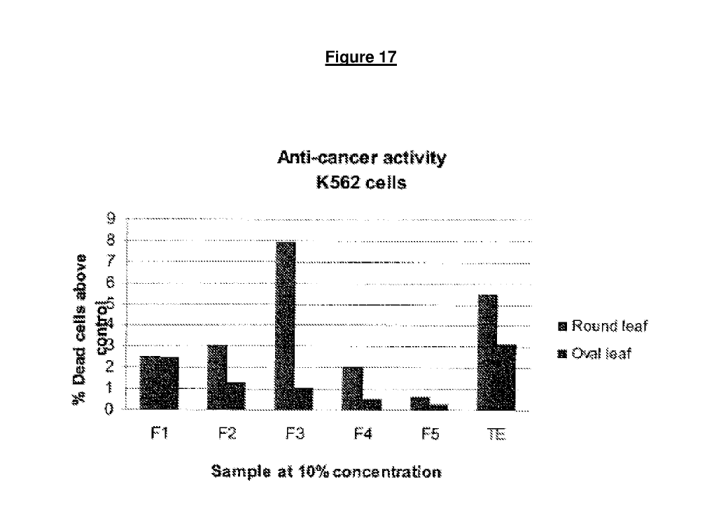

The results are shown in the graphs of the accompanying FIGS. 16 and 17.

The graphs show an effect on Jurkat T cells by Fraction 5 of the oval-leaf time-based extract and a slight effect on K562 cells by Fraction 3 of the round-leaf time-based extract.

Aqueous extracts obtained from Buchu plant material by the process described above can therefore be useful in the treatment of a wide variety of disorders.

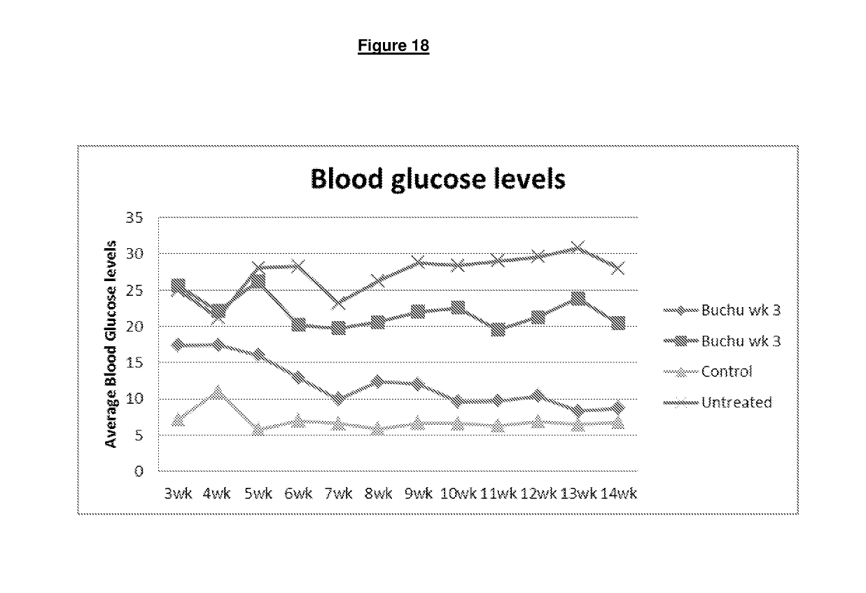

Referring now to FIGS. 18 to 21:

### Experiment 1

To determine whether a watery extract of Buchu will affect glucose homeostasis in a rat model of type 1 diabetes.

The model of type 1 diabetes was created by injecting adult Wistar rats once off with 30 mg/kg streptozotocin to partially ablate the pancreatic beta cells. The blood glucose levels of the animals, measured in a non-fasting state, rose to diabetic levels within 24 hours. As the animals treated in this manner, sometimes spontaneously revert to normal and withstand the effects of the streptozotocin, we allowed the rats to stabilize for 3 weeks. At the end of 3 weeks, the blood glucose levels were again determined and the animals divided into 4 groups: (i) control—blood glucose 7.2±0.5 mmol/L, n=6; (ii) untreated—blood glucose 25±0.8 mmol/L, n=4; (iii) type 1—blood glucose 25.6±1.2 mmol/L, n=5 and (iv) type 1—blood glucose 19.9±2.6 mmol/L, n=5. Groups (iii) and (iv) were both treated with Buchu water from week 3 onwards. FIG. 1 depicts the blood glucose levels followed over a period of 14 weeks.

From FIG. 18 it is evident the all animals treated with Buchu water responded with a lowering of blood glucose levels. It can also be noted that animals with extremely high blood glucose levels, had a lag phase before responding but also ended with significantly lower blood glucose levels. The values given are the mean of all animals in the cage as they were not individually housed.

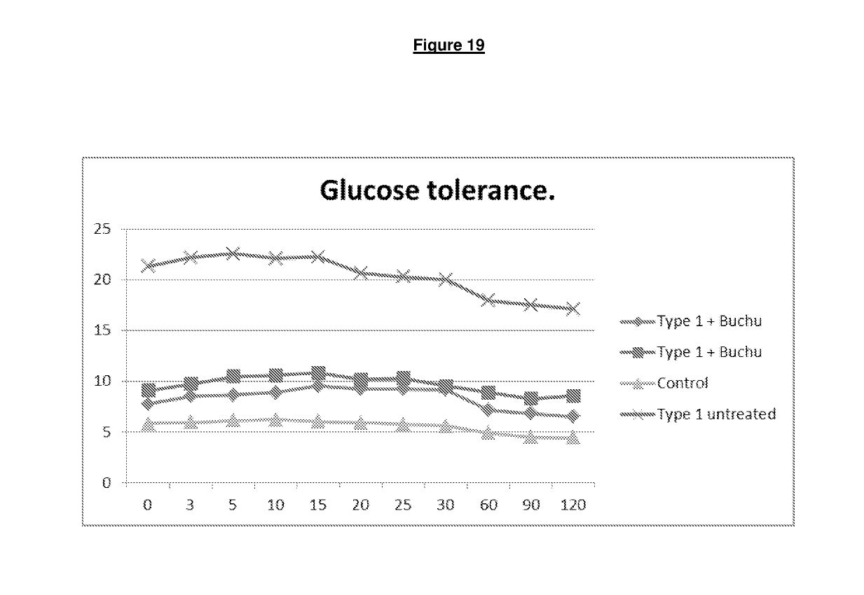

At the end of the 14 week period, the glucose tolerance of the animals was determined as depicted in FIG. 19. This is done after an 18 hour fast by injecting the animals intra-peritoneally once-off with 1 mg/kg sucrose and following the blood glucose levels at set time intervals over a 2-hour period (IPGTT'S). As these curves are generated from the fasting state, they give a better picture of the whole-body insulin resistance in the animals. It must be kept in mind that these animals all had severely curtailed pancreatic function to start with.

In FIG. 19 it can clearly be seen that both groups of type 1 diabetic animals that were treated with Buchu water presented with curves comparable to control animals. The type 1+Buchu group indicated by the red line started off with extremely high blood glucose values and ended with a slight but significantly higher 2-hour value than control animals while the group indicated by the blue line presented with curves not significantly different from control (1-way ANOVA).

Accordingly the Buchu water had a profound effect on the blood glucose levels of all the type 1 diabetic animals treated.

### Experiment 2

To determine whether a watery extract of Buchu will a) affect glucose homeostasis in a rat model of type 2 diabetes and b) protect the heart from infarction

To create a rat model of type 2 diabetes is quite difficult as rats, even on a high fat diet, do not become diabetic as they do not develop hyperglycaemia. The model that we used and characterized in the past is typical pre-diabetic with dyslipidaemia and high insulin levels but normal plasma glucose. Accordingly the model was changed by keeping the rats on a diet to induce obesity and dyslipidaemia for a period of 8 weeks and then injecting them with a low dose of streptozotocin (30 mg/kg) to ablate 50% of the pancreatic beta cells. Apparently this combination of high fatty acids as well as high glucose levels is quite lethal as we lost many animals! The animals in the this part of the project have been divided into the following groups: (i) control; (ii) control+Buchu water; (iii) Diabetic; (iv) Diabetic+Buchu water from day 1; (v) Diabetic+Buchu water from week 8; (vi) Diabetic+Metformin as positive control.

### Experiment 3

Description: 1 sample of Buchu oil and 1 sample of Buchu water

Product Description: The Buchu oil sample is produced by a steam distillation process and the Buchu water is a by-product of this process.

Test Description: Anti-glycemic activity

Products—Composition Sample ID

Buchu Oil CK_Oil_060611

Buchu water CK_H₂O_060611

Preparation—The buchu oil was diluted with methanol to yield a stock with a concentration of 600 μg/ml. The buchu oil was tested at final concentrations of 0.6, 0.3 and 0.15 μg/ml (dilutions made with distilled H₂O). The buchu water was diluted with distilled water to obtain required concentrations. The buchu water was tested at final concentration of 150, 300 and 600 times diluted. The concentrations used closely correspond to the theoretical double physiological dose, physiological dose and half of physiological dose.

Procedure—The product was tested against Chang liver and 3T3-L1 adipose cells using a glucose utilization assay. One micro-molar (μM) Insulin and 1 μM Metformin was used as positive controls for the 3T3-L1 and the Chang liver cells respectively. Cells were cultured to sufficient numbers in appropriate growth media, where after they were fed with fresh media and incubated in the presence of the buchu preparations or the positive controls. Glucose utilization by the cells in the presence of the buchu preparations were compared to that of the positive and negative controls (cells only). An increase in glucose utilization would indicate possible anti-glycemic potential of the relevant test sample.

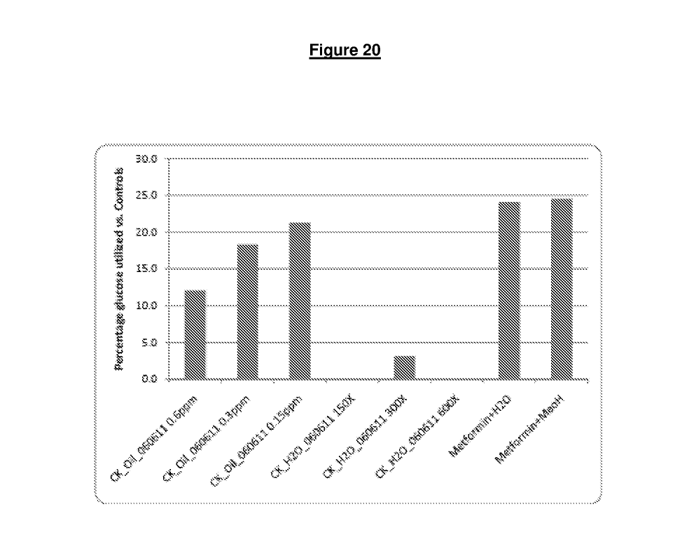

A commercial glucose assay kit was used to determine the glucose concentration in the supernatants of the various samples. Initial screening of the 3T3-L1 cells showed that the glucose concentration was high in these samples and therefore samples were diluted to fall within the range of the assay. The results are shown in FIG. 20.

Percentage glucose utilization compared to the negative controls was calculated as follow:

$$\% glucose utilization = \frac{Negative control - test sample}{Negative control} \times 100$$

Discussion—The buchu oil displayed anti-glycemic activity in the Chang liver cell cultures (FIG. 20). The fact that these cells are sensitive to Metformin suggests that the mode of action of the buchu oil could be similar to that of the small molecule Metformin. The buchu water did not display any significant activity in the Chang liver cell cultures.

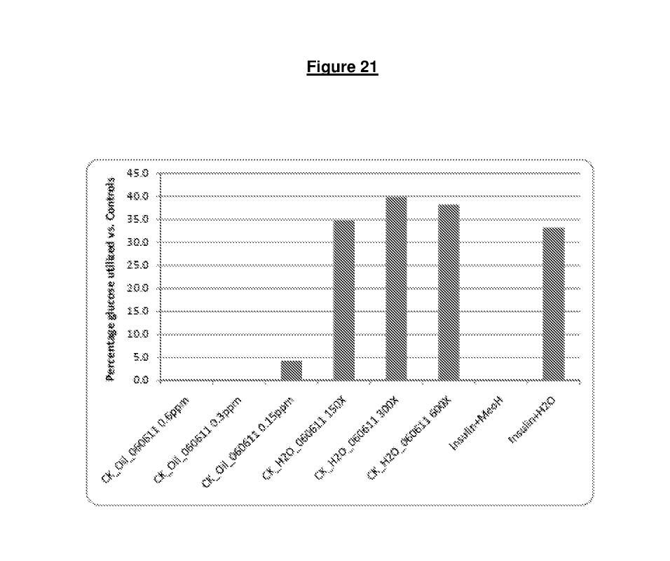

Only the buchu water demonstrated activity in 3T3-L1 cell cultures (FIG. 21). These cells are sensitive to the hormone Insulin; therefore any regulation on glucose utilization would be receptor-mediated.

The results suggest that the buchu products exhibit a reasonable anti-glycemic activity, although not as potent as the respective positive controls. Furthermore the results suggest that there are different components in the buchu oil and the buchu water that are responsible for the observed activity, but this will have to be confirmed by further studies.

Aqueous extracts obtained from Buchu plant material by the process described above can therefore be useful in the treatment of a wide variety of disorders.

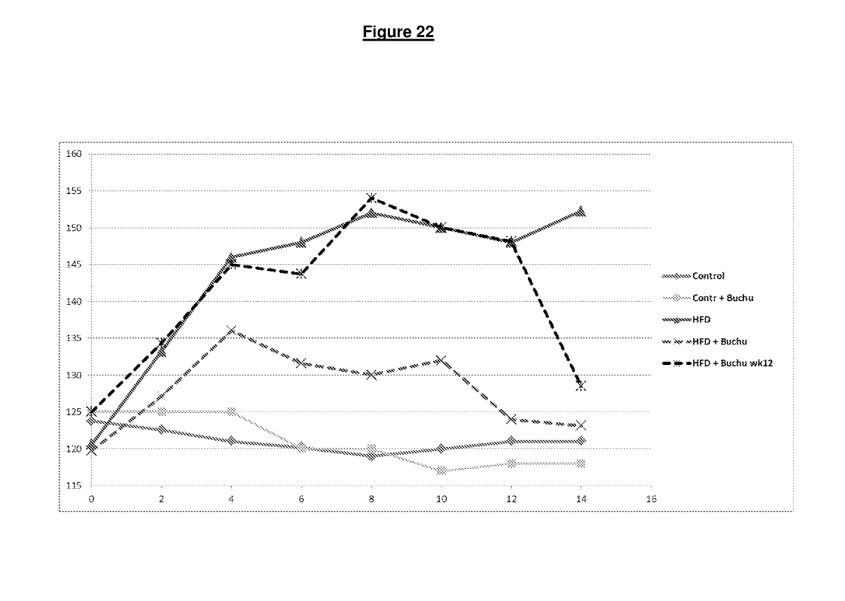

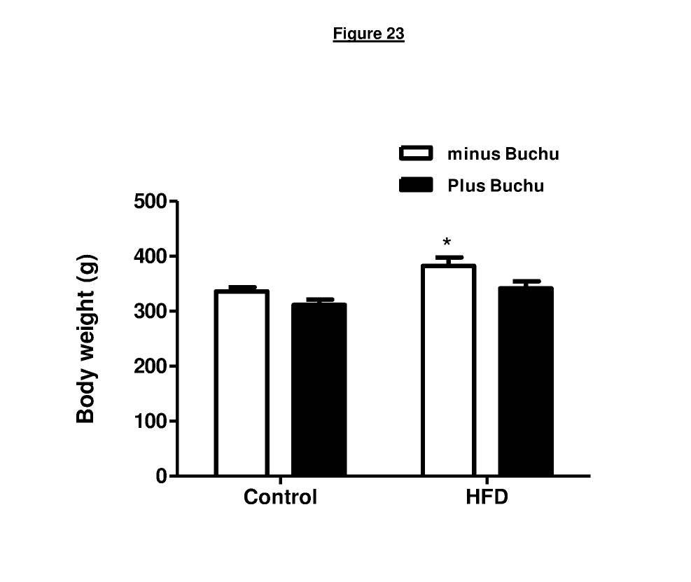

Referring now to FIGS. 22 and 23, as mentioned in the background to the invention, the oil extracted from Buchu plant material has traditionally been used in commercial therapeutic and other uses. It has now surprisingly been found that Buchu plant material includes a wealth of water soluble molecules or components that have been found to show various therapeutic properties including, but not necessarily exclusively, anti-bacterial, anti-fungal, anti-inflammatory and anti-cancer properties.

### Experiment 1

To determine whether a watery extract of Buchu will affect the blood pressure levels in a rat model of hypertension.

To create hypertensive animals, Wistar rats are fed a diet containing 40% animal fat and supplemented with cholesterol, casein and fructose (HFD). The blood pressure of these animals become significantly higher than animals fed normal rat chow within 2 weeks of starting on the respective diets. To answer the question posed, the blood pressure of the following groups of animals were followed on a weekly basis: (i) control chow fed; (ii) control chow fed+Buchu water; (iii) HFD; (iv) HFD+Buchu water from day 1; (v) HFD+Buchu water from week 12.

FIG. 22 shows the effects of both the HFD and the Buchu treatment up to week 14. This experiment was terminated at week 16.

The Buchu water had profound effects on blood pressure development as well as the treatment of high blood pressure. As can be seen in FIG. 22, animals that received the Buchu water treatment in conjunction with the HFD, did not develop hypertension while hypertensive animals that were treated with the Buchu water (the dotted black line) are in the process of normalizing their blood pressure.

### Effects on Body Weight

In the course of the experimental time, animals have also been weighed at various time-points. It was observed that both control chow fed animals as well as animals on the HFD that was treated with Buchu water, presented with a lower mean body weight. The weight of the animals was not followed on an individual basis, therefore the values represent the mean of the whole experimental group. There was a significant effect of the Buchu water treatment on weight loss as indicated by a 2-way ANOVA and shown in the graph below with p<0.01, n=10 per group (*=p<0.05 control vs HFD). It might be a good idea to extend this result by monitoring the weight of individual animals over a period of time! In addition, we did not measure food consumption of the animals and that would be necessary if one should wish to make a statement about weight loss.

Aqueous extracts obtained from Buchu plant material by the process described above can therefore be useful in the treatment of a wide variety of disorders.

1. A therapeutic composition, which includes at least one active ingredient originating from an aqueous Buchu extract and/or Buchu oil (betulina agathosma) or bioactive fraction thereof in a pharmaceutically acceptable form.

2. The composition as claimed in claim 1, which is a pharmaceutical composition including a therapeutically effective amount of the at least one active ingredient and one or more pharmaceutically acceptable carriers or additives.

3. The composition as claimed in claim 1, which includes an effective amount of one or more anti-bacterial, anti-fungal, anti-inflammatory, anticancer, or other therapeutic active ingredients.

4. The composition as claimed in claim 1, which is adapted to be used for inducing an anti-bacterial, anti-fungal, antiinflammatory, anti-cancer, or other therapeutic response in a mammal, preferably a human, in need thereof.

5. The composition as claimed in claim 1, which is adapted to be used for treating an ailment, infection or disease in a patient comprising administering to a patient in need thereof a therapeutically effective amount of at least one active ingredient obtained from an aqueous Buchu extract and/or Buchu oil (betulina agathosma) or bioactive fraction thereof.

6. The composition as claimed in claim 1, which is adapted to be used to treat and/or inhibit and/or killing of at least one of the following bacterial strains and/or Fungus:
(a) MRSA;
(b) Pseudomonas aeruginosa;
(c) Shigella;
(d) Staphylococcus aureus (both methicillin and vancomycin resistant strains);
(e) Tuberculosis;
(f) Acinetobacter spp (particularly baumannii);
(g) Vancomycin resistant enterococci;
(h) Klebsiella pneumonia;
(i) Streptococcus pneumonia; and
(j) Malassezia pachydermatis (Fungus).

7. A modified aqueous Buchu extract and/or Buchu oil (betulina agathosma) or bioactive fraction thereof, which includes an effective amount of one or more anti-bacterial, anti-fungal, anti-inflammatory, anti-cancer, or other therapeutic active ingredients Preferably, the aqueous Buchu extract is further treated to obtain one or more bioactive fractions containing at least one active ingredient suitable for use in therapy.

8. The modified aqueous Buchu extract and/or Buchu oil (betulina agathosma) or bioactive fraction thereof as claimed in claim 7, in which the aqueous Buchu extract is contacted with a suitable solvent to obtain various fractions thereof and/or the active ingredients directly.

9. The modified aqueous Buchu extract and/or Buchu oil (betulina agathosma) or bioactive fraction thereof as claimed in claim 7, in which the aqueous extract is obtained from water-based by-product(s) or residue(s) from a conventional Buchu oil preparation process, the water-based byproducts) or residue(s) being further treated to recover the aqueous Buchu extract.

10. The modified aqueous Buchu extract and/or Buchu oil (betulina agathosma) or bioactive fraction thereof as claimed in claim 8, in which the solvent is selected based on its polarity to extract active molecules from the aqueous Buchu extract.

11. The modified aqueous Buchu extract and/or Buchu oil (betulina agathosma) or bioactive fraction thereof as claimed in claim 10, in which the solvents include ethyl acetate (EtAc), dichloromethane (DCM) and chloroform (chlfm), and/or chloroform.

12. The therapeutic composition of claim 1, wherein the pharmaceutically acceptable form comprises a watery extract of at least one active ingredient originating from an aqueous Buchu extract or bioactive fraction thereof in a pharmaceutically acceptable form.

13. The composition as claimed in claim 12, which is a pharmaceutical composition comprising a water extract of a therapeutically effective amount of at least one active ingredient and one or more pharmaceutically acceptable carriers or additives.

14. The therapeutic composition, which includes Buchu plant material extracts and which is adapted for treating Type 1 and Type 2 diabetes.

15. A modified aqueous Buchu extract or bioactive fraction thereof, which includes an effective amount of one or more anti-diabetic therapeutic active ingredients, an effective amount of one or more anti-hypertensive therapeutic active ingredients and/or an effective amount of one or more anti-bacterial, anti-fungal, anti-inflammatory, anti-cancer, or other therapeutic active ingredients.

16. (canceled)

17. (canceled)

18. A method of treating diabetes in a patient, which includes the step of administering to a patient in need thereof a therapeutically effective amount of at least one active ingredient obtained from an aqueous Buchu extract or bioactive fraction thereof.

19. A method of preparing an aqueous Buchu extract, which includes the steps of providing a source of Buchu plant material, preferably from the species Agasthoma betulina (round-leaf Buchu) and/or Agathosma crenulata (oval-leaf Buchu), treating the plant material in order to extract the water soluble components thereof into an aqueous portion, and treating the aqueous portion to remove any non-water-soluble components and/or other impurities.

20. The method as claimed in claim 19, in which the aqueous Buchu extract is further treated to obtain one or more bioactive fractions containing at least one active ingredient suitable for use in therapy.

21. The method as claimed in claim 18, in which the aqueous Buchu extract is contacted with a suitable solvent to obtain various fractions thereof and/or the active ingredients directly.

22. The method as claimed in claim 18, in which the aqueous extract is obtained from the water-based by-product(s) or residue(s) from a conventional Buchu oil preparation process, the water-based by-product(s) and/or residue(s) being further treated to recover the aqueous Buchu extract.

23. The method as claimed in claim 21, in which the solvent is selected based on its polarity to extract active molecules from the aqueous Buchu extract.

24. The method as claimed in any one of claim 21, in which the solvents include ethyl acetate (EtAc), dichloromethane (DCM) and chloroform (chlfm), and/or chloroform.

25. (canceled)

26. A method of treating hypertension in a patient, which includes administering to a patient in need thereof a therapeutically effective amount of at least one active ingredient obtained from an aqueous Buchu extract or bioactive fraction thereof.

27-33. (canceled)

34. A method of treating an ailment, infection or disease in an animal, which includes the step of administering to an animal in need thereof a therapeutically effective amount of at least one active ingredient obtained from an aqueous Buchu extract and/or buchu oil (betulina agathosma) or bioactive fraction thereof.

35-42. (canceled)
